(19) United States
(12) Patent Application Publication
(10) Pub. No.: US 2012/0046183 A1
(43) Pub. Date: Feb. 23, 2012

(54) METHOD OF DIAGNOSING A MENTAL STATE

(75) Inventors: Johannes Hendrick Van Heerden, Amsterdam (NL); Dan Joseph Stein, Cape Town (ZA); Nicola Illing, Cape Town (ZA)

(73) Assignee: University of Cape Town, Cape Town (ZA)

(21) Appl. No.: 13/202,716

(22) PCT Filed: Feb. 22, 2010
(86) PCT No.: PCT/IB10/50768

§ 371 Date: Nov. 1, 2011

(30) Foreign Application Priority Data
Feb. 20, 2009 (ZA) 2009/01221

(51) Int. Cl. C40B 30/04; C12Q 1/68

(52) U.S. Cl. 506/9; 435/6.12; 435/6.11

(57) ABSTRACT

A method of assessing a psychiatric disorder, behavioural problem or mental state following exposure to stress is described. Expression levels of mRNA transcripts for a plurality of genes linked to a stress-related neural state in the peripheral blood of a subject are measured. The expression levels of the genes, relative to a reference set of expression levels for the same genes in a healthy subject, are used to predict or assess a psychiatric disorder following exposure to a stressor.

### BACKGROUND OF THE INVENTION

This invention relates to a method of assessing a neuropsychiatric state, psychiatric disorder, behavioural problem or mental state in a subject by detecting the expression level of mRNA transcripts for a set of genes linked to a stress-related neural state in that subject.

The prevalence of neuropsychiatric disorders is assuming pandemic proportions and are projected to become some of the most important contributors to the world's disease burden within the next two decades.

The treatment of psychiatric disorders and the efficiency of clinical intervention strategies have to a large extent been limited by the effectiveness of diagnostic techniques. Diagnoses of psychiatric disorders rely heavily on the subjective categorical identification of discrete symptom clusters. These categorical approaches have important drawbacks as psychopathological conditions are classified on the basis of observed symptoms. The fact that most psychiatric disorders are progressive, not showing classifiable symptoms during early stages of onset, makes efficient diagnosis and early intervention difficult. The problem is further compounded by overlapping clinical features between many common disorders. Although the neurobiology of many psychiatric disorders is fairly well understood, direct or invasive interrogation of the neural milieu is, for obvious reasons, not practical.

It would be useful to have a non-invasive (or indirect) method of interrogating neural states for use as a psychiatric assessment and diagnostic tool, especially for assessing or diagnosing psychiatric disorders, behavioural problems or mental states following exposure to stress.

### SUMMARY OF THE INVENTION

According to a first embodiment of the invention, there is provided a method of assessing a psychiatric disorder, behavioural problem or mental state in a subject following exposure to stress, the method including the steps of:
(a) determining the expression levels of at least 80% of the genes listed in Table 1 in a sample from the subject; and
(b) comparing the expression levels of these genes with a reference set of expression levels for the same genes, wherein a difference in the expression levels in the subject and the reference set is indicative of whether the subject has, or is susceptible to developing, a psychiatric disorder, behavioural problem or mental state.

The reference set of expression levels may comprise expression levels of the genes of a healthy or normal individual (i.e. an individual who does not have, or is not susceptible to developing, a psychiatric disorder, behavioural problem or mental state).

An algorithm may be used to compare the expression levels of the subject with the reference set and based on the comparison, to predict whether the subject has, or is susceptible to developing, the psychiatric disorder, behavioural problem or mental state.

In step (a), expression levels of at least 90%, at least 95% or all of the genes listed in Table 1 may be determined in the sample from the subject. Expression levels of genes other than those in Table 1 may also be determined in the sample from the subject. The other genes may be selected from those listed in Table 2, and expression levels of at least 80%, at least 90%, at least 95% or all of the genes listed in Table 2 may be determined in step (a).

The method may be performed by detecting levels of mRNA transcripts encoded by the genes of Tables 1 and/or 2.

The sample may be a bodily sample containing white blood cells, such as a blood or buccal smear.

Peripheral blood mononuclear cells (PBMCs) may be isolated from the sample for step (a), and RNA from the PBMCs may be additionally isolated.

Step (a) may be performed using quantitative reverse-transcriptase polymerase chain reaction (RT-qPCR), and in particular by Real-Time PCR. Alternatively, step (a) may be performed using a dot blot procedure, such as a miniaturised dot blot. Alternatively, the method of measuring mRNA transcript abundance may be performed using a microarray printed with oligonucleotides corresponding to at least 80% of the genes listed in Tables and/or 2.

In particular, the method may include the steps of:
(a) isolating PBMCs from whole blood of a subject;
(b) purifying total RNA from the PBMCs;
(c) amplifying mRNA transcripts of the genes selected from Table 1 and/or Table 2 by RT-qPCR;
(d) quantifying the amplified mRNA transcripts;
(e) assessing the relative abundance of the amplified mRNA transcripts of each of the genes; and
(f) predicting patient status using collected mRNA transcript abundances which, in turn, is subjected to classification and prediction algorithms for the grouping of subjects into normal, i.e. healthy, or pathological groups.

Alternatively, the method may include the steps of:
(a) isolating PBMCs from whole blood of a subject;
(b) purifying the total RNA from the PBMCs;
(c) converting the RNA into cDNA by reverse transcription and labelling it with a fluorescent dye or chromagenic substrate;
(d) hybridizing the labelled cDNA to a dot blot printed with probes corresponding to at least 80% of the genes listed in Tables 1 or 2;
(e) quantifying the intensity of the hybridized labelled cDNA, the intensity of the indicator means corresponding to the relative abundance of mRNA transcript; and
(f) predicting patient status using collected mRNA transcript abundances which, in turn, is subjected to classification and prediction algorithms for the grouping of subjects into normal, i.e. healthy, or pathological groups.

Alternatively, the method may include the steps of:
(a) isolating PBMCs from whole blood of a subject;
(b) purifying total RNA from the PBMCs;
(c) converting RNA into cDNA by reverse transcription, and labelling the cDNA with fluorescent dye;
(d) contacting a microarray slide printed with oligonucleotide probes representing genes shown in Table 1 and/or Table 2 with the labelled cDNA;
(e) hybridizing the fluorescent labelled cDNA against the oligonucleotide probes representing genes shown in Table 1 and/or Table 2 printed on the microarray slide quantifying the intensity of fluorescence of cDNA fluorescent probes bound to their target oligonucleotides on the microarray slide, the intensity of fluorescence at each target corresponding to the relative abundance of mRNA transcript for each gene; and
(f) predicting patient status using collected mRNA transcript abundances which, in turn, is subjected to classification and prediction algorithms for the grouping of subjects into normal, i.e. healthy, or pathological groups.

According to a second embodiment of the invention, there is provided a kit for assessing a psychiatric disorder, behavioural problem or mental state following exposure to stress in a subject according to the method described above, the kit including:
(a) (i) primers for amplification of mRNA transcripts by RT-qPCR of at least 80% of the genes listed in Table 1 and/or Table 2;
   (ii) probes printed on a membrane for hybridizing mRNA transcripts of at least 80% of the genes listed in Table 1 and/or Table 2, for use in an RNA dot blot procedure; or
   (iii) oligonucleotides corresponding to the genes listed in Table 1 and/or Table 2, printed on a glass slide, to which fluorescent cDNA prepared from RNA purified from PBMCS can be hybridized when a microarray is used in the method;
      and
(b) an indicator means.

The kit may additionally comprise instructions for performing the method of described above.

The kit may be for performing the method described above using an RNA dot blot process, such as a miniaturised dot blot, a microarray platform and/or RT-qPCR.

The kit may further include a reference set of expression levels for at least 80% of the genes of Table 1 and/or Table 2, wherein the reference set of expression levels comprises expression levels of the genes from a healthy or normal subject.

The kit may also include computer readable instructions for:
(a) comparing the expression levels of the genes from a subject with a reference set of expression levels for the same genes, and
(b) predicting whether the subject has, or is susceptible to developing, a psychiatric disorder, behavioural problem or mental state.

### BRIEF DESCRIPTION OF THE FIGURES

FIG. 1: Differential gene expression results. Venn diagrams show the overlap between different gene selection criteria (Info and SAM P<0.05 and Fold difference >1.2) for (A) PBMC, (B) pFC, (C) Hic and (D) Hyp. This gene selection strategy significantly reduced the number of genes identified as DE by any one single criterion. Also shown are the false colour sample profiles of hierarchically clustered differentially expressed genes for (E) PBMC samples [347 over- and 71 under-expressed], and neural tissues (F) pFC [66 over- and 88 under-expressed], (G) Hic [71 over- and 75 under-expressed] and (H) Hyp [69 over- and 81 under-expressed]. The selected genes produce a clear separation between MS and SH samples. Genes more highly expressed in MS samples are at the top and those more highly expressed in SH samples at the bottom. P=PBMC; F=pFC

FIG. 2: FatiScan gene set enrichment results. Shown are significant co-ordinately expressed GO terms within whole gene sets for (A) PFC and (B) Hic. The normalized percentage of genes annotated with a specific term is indicated for each group. Red indicates coordinated over-expression in MS group and Blue coordinated over-expression SH group (or under-expression in MS group). Colour intensity denotes how strongly a term is over- or under-expressed.

FIG. 3: FatiScan gene set enrichment results. Shown are significant co-ordinately expressed GO terms within whole gene sets for (A) Hyp and (B) PBMC. The normalized percentage of genes annotated with a specific term is indicated for each group. Red indicates coordinated over-expression in MS group and Blue coordinated over-expression SH group (or under-expression in MS group). Colour intensity denotes how strongly a term is over- or under-expressed.

FIG. 4: Schematic summary of neural gene expression results in support of a stress-related hyperglutamatergic state in MS brain samples. Such a hyperglutamatergic state could potentially result in elevated stress-induced corticosterone responses. Red indicates over-expression and blue under-expression, in MS samples, respectively. An asterisk indicates genes or functional classes that were found to be regulated in a coordinated manner. Glu=Glutamate, (+) indicates increased signalling activity, (−) indicates decreased signalling/activity.

FIG. 5. Sample classification and prediction results. (A) Leave-one-out error rates of classifiers. The KNN algorithm (blue line) reaches an optimal prediction efficiency of 95% with a minimum of 50 genes. Using 125 genes the SVM algorithm (green line) obtains this efficiency, and converges with KNN. (B) Hierarchically sample clustered (Pearson correlation metric with average linkage) profiles for the 50 gene predictor set. Notice, that although only 19 out of 20 samples were correctly classified, hierarchical clustering separates all samples into two general treatment-related clusters. (C) A summary of KNN sample classification results, showing details of the misclassification of individual samples. Although most samples classes were correctly predicted, PBMC69, an SH sample, was consistently misclassified. P=PBMC

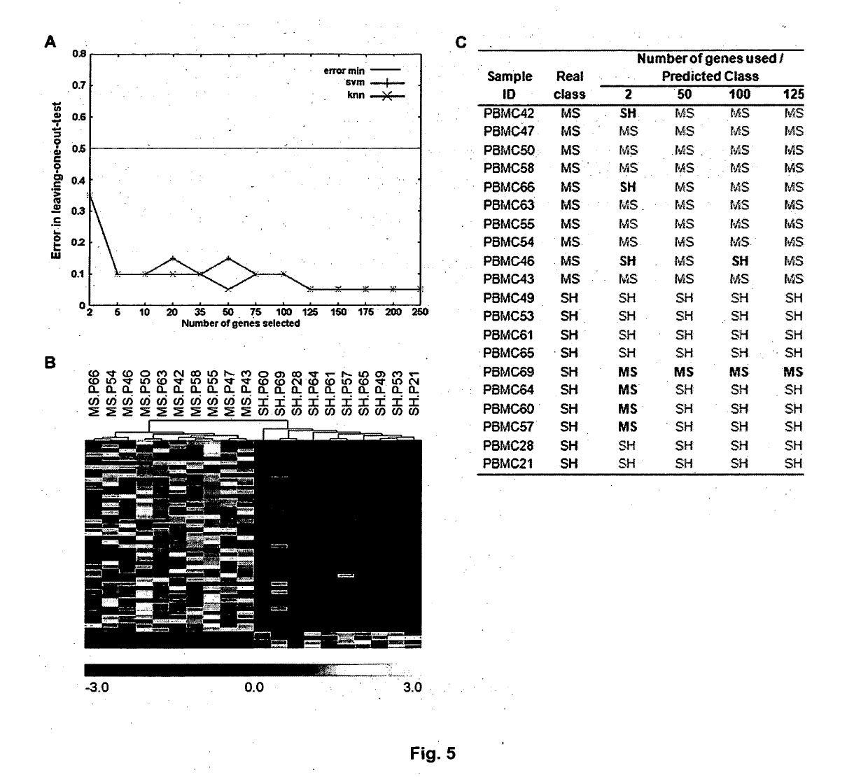

### DETAILED DESCRIPTION OF THE INVENTION

This invention provides a method of assessing or diagnosing a neuropsychiatric state, psychiatric disorder, behavioural problem or mental state following exposure to stress by detecting the expression level of mRNA transcripts for a plurality of genes linked to a stress-related neural state in the peripheral blood of a subject.

As used herein, the term “psychiatric disorder” refers to any disturbance of the mind, or a behavioural problem, or an abnormal mental state. Psychiatric disorders are classified in manuals such as the Diagnostic and Statistical Manual of Mental Disorders (DSM) and the ICD-10 (e.g. schizophrenia, bipolar disorder and major depression).

Relevant stressors include, but are not limited to, early childhood stressors, severe traumas (such as those leading to PTSD) and everyday stressors (such as those that might precipitate a psychiatric disorder in a vulnerable person).

The applicant used the model of maternal separation, which is known to induce long term alterations in neurophysiology and stress-related behaviours in adult rodents to investigate i) whether parallel changes occur in gene expression in three brain regions (the prefrontal cortex, hippocampus, and hypothalamus) and PBMCs and ii) whether gene expression changes in PBMCs could be used to predict the animal treatment group. Two populations were defined, a sample population (SAMPLE), which was subjected to maternal separation during development, and a control population (CONTROL).

Upon reaching adulthood, both populations were evaluated with regards to spontaneous exploration behaviour, in addition to fear-induced corticosterone responses, as a means of evaluating fear- and anxiety profiles of the two populations (van Heerden et al., Behavioural Brain Research 207 (2010) 332-342). Next, animals were killed by means of cervical dislocation, after which trunk blood was collected and the mRNA populations of each sample was relatively quantified.

Microarray gene expression profiles of all three brain regions provided substantial evidence of stress-related neural differences between maternally separated and control animals. For example, changes in expression of genes involved in the glutamatergic and GABAergic systems were identified in the PFC and Hic, supporting a stress-related hyperglutamatergic state within the separated group. The expression of 50 genes (Table 1) selected from the PBMC microarray data provided sufficient information to predict treatment classes with 95% accuracy. A larger set of 125 genes (Table 2), containing the 50 genes of the smaller set, proved to be equally effective. Importantly, stress-related transcriptome differences in PBMC populations were paralleled by stress-related gene expression changes in CNS target tissues.

These results confirm that the transcriptional profiles of peripheral immune tissues occur in parallel to changes in the brain and contain sufficient information for the efficient diagnostic prediction of stress-related neural states in mice.

TABLE 1
50 Genes for assessing or diagnosing whether patients who have been exposed to stress are at risk
of suffering from one or more psychiatric disorders, which classified samples with 95% accuracy

| Operon | | | ENSEMBL/Refseq/ | Over/Under |
|---|---|---|---|---|
| Oligo ID | Description | Symbol | Riken ID | expressed in MS |
| M400008627 | RIKEN cDNA 4921528I07 gene | 4921528I07Rik | ENSMUSG00000074149 | over |
| M200012683 | Acetyl-Coenzyme A | Acat2 | ENSMUSG00000023832 | over |
| | acetyltransferase 2 | | | |
| M400004596 | A disintegrin-like and | Adamts9 | ENSMUSG00000030022 | over |
| | metalloprotease with | | | |
| | thrombospondin type 1 motif, 9 | | | |
| M200000582 | Adenylate cyclase 8 | Adcy8 | ENSMUSG00000022376 | over |
| M200005645 | Actin related protein 2/3 complex, | Arpc5l | ENSMUSG00000026755 | over |
| | subunit 5-like | | | |
| M200006901 | ATPase, H+ transporting, | Atp6v0e2 | ENSMUSG00000039347 | over |
| | lysosomal V0 subunit E2 | | | |
| M400004024 | cDNA sequence BC013672 | BC013672 | ENSMUSG00000037921 | over |
| M400008030 | Bone gamma-carboxyglutamate | Bglap-rs1 | ENSMUSG00000074489 | over |
| | protein, related sequence 1 | | | |
| M300011602 | Carbonic anhydrase 14 | Car14 | ENSMUSG00000038526 | over |
| M200000995 | Cholecystokinins precursor | Cck^{§} | ENSMUSG00000032532 | over |
| M200013753 | Coronin 7 | Coro7 | ENSMUSG00000039637 | over |
| M200003934 | Cytochrome P450, family 2, | Cyp2c29 | ENSMUSG00000003053 | over |
| | subfamily c, polypeptide 29 | | | |
| M300013894 | RIKEN cDNA D130054N24 gene | D130054N24Rik | ENSMUSG00000042790 | over |
| M400003995 | RIKEN cDNA D330050I23 gene | D330050I23Rik | ENSMUSG00000072569 | over |
| M300010488 | Dermokine | Dmkn | ENSMUSG00000060962 | over |
| M200003607 | Dedicator of cytokinesis 7 | Dock7 | ENSMUSG00000028556 | over |
| M300014949 | Endothelial differentiation, | Edg5 | ENSMUSG00000043895 | over |
| | sphingolipid G-protein-coupled | | | |
| | receptor, 5 | | | |
| M400001692 | Predicted gene | EG620592 | ENSMUSG00000071719 | over |
| M400010593 | Forkhead box protein R1 | Foxr1 | ENSMUSG00000074397 | over |
| | (Forkhead box protein N5) | | | |
| M300000132 | Homeo box A4 | Hoxa4 | ENSMUSG00000000942 | over |
| M400013298 | LSM14 protein homolog A (Rap55) | Lsm14a | ENSMUSG00000066568 | over |
| M400004821 | Lysocardiolipin acyltransferase | Lycat | ENSMUSG00000054469 | over |
| M400009939 | Mitogen-activated protein kinase | Map3k9 | ENSMUSG00000042724 | over |
| | kinase kinase 9 | | | |
| M300007290 | Mesoderm posterior 2 | Mesp2 | ENSMUSG00000030543 | over |
| M200007123 | Muted protein | Muted^{§} | ENSMUSG00000038982 | under |
| M200010626 | Matrix-remodelling associated 8 | Mxra8 | ENSMUSG00000073679 | over |
| M200007448 | Nitric oxide synthase interacting | Nosip | ENSMUSG00000003421 | over |
| | protein | | | |
| M300018063 | Olfactory receptor 1495 | Olfr1495 | ENSMUSG00000047207 | over |
| M300017588 | Olfactory receptor 66 | Olfr66 | ENSMUSG00000058200 | over |
| M300015973 | Olfactory receptor 669 | Olfr669 | ENSMUSG00000073916 | over |
| M300002331 | Predicted gene | MGI: 3652048 | ENSMUSG00000020682 | over |
| M200003458 | Oxytocin | Oxt | ENSMUSG00000027301 | over |
| M400010890 | Mus musculus polymerase (RNA) II | Polr2c | ENSMUSG00000031783 | over |
| | (DNA directed) polypeptide C | | | |
| M200000936 | Peripherin 1 | Prph1 | ENSMUSG00000023484 | over |
| M300003403 | PTK2 protein tyrosine kinase 2 | Ptk2 | ENSMUSG00000022607 | under |
| M400001722 | Slingshot homolog 3 (Drosophila) | Ssh3 | ENSMUSG00000034616 | over |
| M300003482 | Type 2 lactosamine alpha-2,3- | St3gal6^{§} | ENSMUSG00000022747 | under |
| | sialyltransferase | | | |
| M200000227 | Stromal interaction molecule 1 | Stim1 | ENSMUSG00000030987 | over |
| M300001453 | Surfeit gene 5 | Surf5 | ENSMUSG00000015776 | over |
| M400000616 | Thrombopoietin precursor | Thpo | ENSMUSG00000022847 | over |
| M400009774 | Transmembrane BAX inhibitor | Tmbim1 | ENSMUSG00000006301 | over |
| | motif containing 1 | | | |
| M200013582 | Transmembrane protein 25 | Tmem25 | ENSMUSG00000002032 | over |
| M400000938 | Transmembrane protein 63A | Tmem63a^{§} | ENSMUSG00000026519 | under |
| M400013169 | Xin actin-binding repeat containing | Xirp2 | ENSMUSG00000027022 | over |
| | 2 isoform 2 | | | |
| M400014435 | Zinc finger protein 84 | Zfp84 | ENSMUSG00000046185 | over |
| M400018008 | Novel Protein | Not assigned | AC160535 | over |
| M400012711 | Novel protein (I830077J02Rik) | Not assigned | AC121847 | over |
| M400017112 | Uncharacterised | Not assigned | AK054246 | over |
| M400003712 | Uncharacterised | Not assigned | AC122270 | Over |
| M400008575 | Uncharacterised | Not assigned | ENSMUSG00000064159 | Over |

TABLE 2
Genes for assessing or diagnosing whether patients
who have been exposed to stress are at risk of suffering
from one or more psychiatric disorders

| ENSEMBL GENE ID | |
|---|---|
| (http://www.ensembl.org/ | |
| index.html) | Gene Name |
| ENSMUSG00000027301 | oxytocin |
| ENSMUSG00000060962 | dermokine |
| ENSMUSG00000020682 | predicted gene, OTTMUSG00000000934 |
| ENSMUSG00000047207 | olfactory receptor 1495 |
| ENSMUSG00000022376 | adenylate cyclase 8 |
| ENSMUSG00000030987 | stromal interaction molecule 1 |
| ENSMUSG00000026755 | actin related protein 2/3 complex, subunit |
| | 5-like |
| ENSMUSG00000031783 | — |
| ENSMUSG00000023484 | peripherin 1 |
| ENSMUSG00000064159 | — |
| ENSMUSG00000023832 | — |
| ENSMUSG00000039637 | — |
| ENSMUSG00000042790 | RIKEN cDNA D130054N24 gene |
| AC122270 | — |
| AC160535 | — |
| ENSMUSG00000030543 | mesoderm posterior 2 |
| ENSMUSG00000058200 | olfactory receptor 66 |
| ENSMUSG00000066568 | — |
| AK054246 | — |
| ENSMUSG00000074397 | — |
| ENSMUSG00000002032 | transmembrane protein 25 |
| ENSMUSG00000071719 | — |
| ENSMUSG00000039347 | ATPase, H+ transporting, lysosomal V0 |
| | subunit E2 |
| ENSMUSG00000003053 | cytochrome P450, family 2, subfamily c, |
| | polypeptide 29 |
| ENSMUSG00000003421 | nitric oxide synthase interacting protein |
| ENSMUSG00000034616 | slingshot homolog 3 (Drosophila) |
| ENSMUSG00000043895 | endothelial differentiation, sphingolipid |
| | G-protein-coupled receptor, 5 |
| ENSMUSG00000022747 | ST3 beta-galactoside |
| | alpha-2,3-sialyltransferase 6 |
| ENSMUSG00000046185 | — |
| AC121847 | — |
| ENSMUSG00000000942 | homeo box A4 |
| ENSMUSG00000038526 | carbonic anhydrase 14 |
| ENSMUSG00000054469 | lysocardiolipin acyltransferase |
| ENSMUSG00000073679 | matrix-remodelling associated 8 |
| ENSMUSG00000037921 | cDNA sequence BC013672 |
| ENSMUSG00000026519 | transmembrane protein 63a |
| ENSMUSG00000027022 | — |
| ENSMUSG00000032532 | cholecystokinin |
| ENSMUSG00000074149 | RIKEN cDNA 4921528I07 gene |
| ENSMUSG00000042724 | mitogen-activated protein kinase kinase |
| | kinase 9 |
| ENSMUSG00000006301 | transmembrane BAX inhibitor motif |
| | containing 1 |
| ENSMUSG00000030022 | — |
| ENSMUSG00000022607 | PTK2 protein tyrosine kinase 2 |
| ENSMUSG00000038982 | muted |
| ENSMUSG00000073916 | olfactory receptor 669 |
| ENSMUSG00000074489 | bone gamma-carboxyglutamate protein, |
| | related sequence 1 |
| ENSMUSG00000072569 | RIKEN cDNA D330050I23 gene |
| ENSMUSG00000015776 | surfeit gene 5 |
| ENSMUSG00000028556 | dedicator of cytokinesis 7 |
| ENSMUSG00000022847 | thrombopoietin |
| NM_175192 | — |
| ENSMUSG00000022781 | p21 (CDKN1A)-activated kinase 2 |
| ENSMUSG00000031770 | homocysteine-inducible, endoplasmic |
| | reticulum stress-inducible, ubiquitin-like |
| | domain member 1 |
| ENSMUSG00000066757 | SEC61, gamma subunit |
| ENSMUSG00000018999 | solute carrier family 35, member B4 |
| ENSMUSG00000057157 | — |
| ENSMUSG00000047163 | olfactory receptor 64 |
| ENSMUSG00000021933 | guanylate cyclase 1, soluble, beta 2 |
| ENSMUSG00000017119 | neighbor of Brca1 gene 1 |
| ENSMUSG00000041359 | T-cell lymphoma breakpoint 1 |
| ENSMUSG00000019808 | deaminase domain containing 1 |
| ENSMUSG00000003464 | peroxisome biogenesis factor 19 |
| ENSMUSG00000036892 | proline dehydrogenase (oxidase) 2 |
| ENSMUSG00000023051 | TAR (HIV) RNA binding protein 2 |
| ENSMUSG00000051133 | RIKEN cDNA 1110020P15 gene |
| ENSMUSG00000038871 | 2,3-bisphosphoglycerate mutase |
| ENSMUSG00000001288 | retinoic acid receptor, gamma |
| AK043175 | — |
| ENSMUSG00000045989 | RIKEN cDNA 4930451I11 gene |
| ENSMUSG00000075150 | olfactory receptor 1137 |
| ENSMUSG00000035505 | COX18 cytochrome c oxidase assembly |
| | homolog (S. cerevisiae) |
| ENSMUSG00000016262 | — |
| ENSMUSG00000026887 | mitochondrial ribosome recycling factor |
| ENSMUSG00000068615 | gap junction membrane channel protein |
| | alpha 9 |
| AC114984 | — |
| ENSMUSG00000055697 | predicted gene, ENSMUSG00000055697 |
| ENSMUSG00000032501 | tribbles homolog 1 (Drosophila) |
| ENSMUSG00000030364 | C-type lectin domain family 2, member h |
| AC140381 | — |
| AK011969 | — |
| ENSMUSG00000019734 | leukocyte receptor cluster (LRC) member 1 |
| ENSMUSG00000032485 | SREBF chaperone |
| ENSMUSG00000021024 | proteasome (prosome, macropain) subunit, |
| | alpha type 6 |
| ENSMUSG00000076599 | Immunoglobulin Kappa light chain V gene |
| | segment |
| ENSMUSG00000034730 | brain-specific angiogenesis inhibitor 1 |
| ENSMUSG00000014198 | RIKEN cDNA A930006D11Rik gene |
| AK034409 | — |
| ENSMUSG00000025956 | RIKEN cDNA 2310038H17 gene |
| ENSMUSG00000003865 | — |
| AC139350 | — |
| ENSMUSG00000006218 | — |
| ENSMUSG00000046323 | developmental pluripotency-associated 3 |
| ENSMUSG00000036744 | olfactory receptor 706 |
| ENSMUSG00000064061 | RIKEN cDNA 2310047C04 gene |
| ENSMUSG00000022475 | histone deacetylase 7A |
| ENSMUSG00000042454 | — |
| ENSMUSG00000022564 | glutamate receptor, ionotropic, N-methyl |
| | D-asparate-associated protein 1 |
| | (glutamate binding) |
| ENSMUSG00000071551 | aldo-keto reductase family 1, member C19 |
| ENSMUSG00000027080 | mediator of RNA polymerase II |
| | transcription, subunit 19 homolog (yeast) |
| ENSMUSG00000055110 | RIKEN cDNA A630012P03 gene |
| ENSMUSG00000032194 | ankyrin repeat domain 25 |
| ENSMUSG00000041789 | RIKEN cDNA 2700046A07 gene |
| ENSMUSG00000027346 | preimplantation protein 4 |
| ENSMUSG00000037257 | RIKEN cDNA 2310007F21 gene |
| CT030173 | — |
| ENSMUSG00000037275 | gem (nuclear organelle) associated protein 5 |
| ENSMUSG00000018381 | ABI gene family, member 3 |
| ENSMUSG00000030206 | — |
| ENSMUSG00000006699 | cell division cycle 42 homolog (S. cerevisiae) |
| ENSMUSG00000034623 | — |
| ENSMUSG00000053337 | predicted gene, EG433873 |
| ENSMUSG00000059355 | cDNA sequence BC056474 |
| AC162788 | — |
| ENSMUSG00000070645 | renin 2 tandem duplication of Ren1 |
| ENSMUSG00000030337 | vesicle-associated membrane protein 1 |
| ENSMUSG00000050961 | hematological and neurological |
| | expressed 1-like |
| AK037255 | — |
| AC139942 | — |
| ENSMUSG00000028719 | cytidylate kinase |
| ENSMUSG00000000606 | vomeronasal 2, receptor, 3 |
| ENSMUSG00000002227 | Moloney leukemia virus 10 |
| ENSMUSG00000013539 | Ser/Thr-rich protein T10 in DGCR region. |
| ENSMUSG00000035498 | CUB domain containing protein 1 |
| ENSMUSG00000031844 | hydroxysteroid (17-beta) dehydrogenase 2 |
| ENSMUSG00000069808 | RIKEN cDNA 2310047D13 gene |

The quantification of the expression levels of the genes listed in Table 1 or Table 2 is by measurement of the abundance of mRNA transcripts of the genes by either RT-qPCR, including Real Time RT-PCR, or the miniaturized fluorescent RNA dot blot method (Yadetie et al. (2004) BMC Biotechnol. 10; 4:12) for rapid quantitation of gene expression in a blood sample from a subject expected to be at a risk of developing, or suffering from, the clinical sequelae of exposure to stressors, or suffering a psychiatric disorder or behavioural problem in which stressors or traumas may play a role. The expression levels of the genes can also be detected by other molecular biological, microfluidic or nano-technologies.

The RT-qPCR can be performed using primers specific for the genes listed in Table 1. Alternatively, the RT-qPCR can be performed using a probe specific for amplified product of at least one of the genes. The RT-qPCR can also be performed using an indicator means that contacts double-stranded DNA (dsDNA). The primers or probe may include the indicator means. The dot blot may include a support, such as a membrane or a glass slide.

The indicator means can be a labelled probe, such as a fluorescent probe, a radiolabelled probe, a chromatographic probe or the like. The indicator means can alternatively be a reporter molecule that contacts the primer or the probe, or amplified PCR product. The indicator means can also be one or more labelled primers.

A whole-genome microarray platform can be used to assess and compare the relative expression (in the form of mRNA transcripts) of all currently sequenced mouse genes. Due to the limited quantities of blood obtained from mouse samples, RNA samples were amplified in the experiments described below. This step may not be necessary in human populations, however, where blood samples can be larger. In addition, a globin reduction step can be used in clinical samples, prior to the assessment of mRNA levels. This step will increase the accuracy and sensitivity of detection for all other mRNA transcripts.

After the collection and quantification of expression values for all mRNA populations, data can be prepared for analysis. This analysis can entail the use of classification, discrimination and grouping algorithms, such as those implemented by GEPAS (http://www.gepas.org) (e.g. Support Vector Machine and K-nearest neighbour), which evaluate the data to determine if the mRNA expression levels of any subset of genes can effectively and reproducibly distinguish between SAMPLE and CONTROL populations. This analysis typically generates a prediction model, which consists of a subset of genes which most consistently differ between SAMPLE and CONTROL populations, should such a set exist. This prediction model can subsequently be applied to any sample derived from other populations and be used to classify this sample as either similar to the SAMPLE or the CONTROL populations which were used during the initial classification. Any other alternative strategies that might be suitable as efficient bioinformatic approaches to biological classification and discrimination for the grouping of patients can also be used.

Additional genes to those listed in Table 1 or Table 2 could also be used to predict specific psychiatric states and symptoms (e.g. stressors may lead to depression, anxiety and even psychosis). Additional genes could also be used to assess response to treatment or to predict response to treatment.

Kits can be provided to perform the invention using RT-qPCR, microarrays or dot blot procedures. The RT-qPCR kit could at least include primers for amplification of the mRNA transcripts of a number of genes linked to a stress-related neural state in a sample from the subject, an indicator means and optionally, instructions for use. The dot blot kit could at least include probes for contacting mRNA transcripts of a number of genes linked to a stress-related neural state in a sample from the subject, an indicator means and optionally, instructions for performing the method of the invention.

The method described herein, which provides for assessment and diagnosis based on the expression levels of genes in PBMCs, can aid clinicians by providing an objective and physiological measure of the consequences of stressors and of associated psychobiological states, rather than having to rely on clinical signs and symptoms, which have limited reliability and validity.

The invention is further described in more detail by the following example, which is not to be construed as limiting in any way either the spirit or scope of the invention.

### EXAMPLE

### Materials and Methods

The protocol was approved by the animal ethics committee of the University of Cape Town (Ethics clearance number: 006/007) and is in accordance with national guidelines for the care and use of laboratory animals. Details of the experiments described below have also been published in Van Heerden et al. (BMC Research Notes 2009, 2:195) and Van Heerden et al. (2010), both of which are expressly incorporated herein.

### Animals and Treatment

Female C57BL16 mice were mated in a specified pathogen free (SPF) environment, and transported to the experimental facility at least three days prior to parturition. All animals were maintained under a 12 h light-dark cycle (lights on from 6 h 00 to 18 h 00). Temperature was kept at 21±2° C. Animals had ad libitum access to sterilized food and tap water. All animal-human interactions were limited to a single researcher. Postnatal day (PND) 0 was assigned to litters born before 15 h 30 each day. Litters were randomly assigned to undergo maternal separation (MS; n=10) or to be reared under standard conditions, with simulated handling (control) events (SH; n=9). The average litter size of both MS and SH groups was equal (n=7).

Maternal separation was carried out as described in Romeo et al. (Hormones and Behaviour, 2003, 43(5): 561-567), with some modifications. Briefly, MS litters were separated from dams for 3 h a day, starting at 12 h 00 (6 h after lights on) and ending at 15 h 00, from PND 1 to 14. The MS dams were first removed from the home cage, after which the pups were moved to a clean cage, which was kept at the ambient temperature of the vivarium. The dam was placed back in the home cage and moved to a separate room for the duration of the separation, this to exclude olfactory or ultrasound vocalization exchanges between dams and their pups. After 3 h, pups and dams were reunited in their home cage. SH animals underwent daily handling. SH dams were removed from the home cage; pups were briefly moved to a clean cage and immediately returned to their home cage, followed by the dam. This procedure simulated the handling undergone by MS pups and served as a control, never lasting for more than 5 min per litter. At PND 21, all pups were weaned and group housed by sex and treatment. All subsequent procedures were carried out using males only, as the consequences of separation are gender specific (Romeo et al., 2003).

### Acute Restraint Stress, Sacrifice, Blood Collection and Brain Dissections

Mice (N_{MS}=30, N_{SH}=30) were subjected to 10 min of acute restraint stress and allowed to recover for 20 min prior to sacrifice. All mice were sacrificed, within 15 s of removal from the cage, by means of cervical dislocation, immediately followed by decapitation and collection of trunk blood. Trunk blood was collected into 1.5 ml tubes pre-filled with 100 μl 3.8% (w/v) tri-Sodium-Citrate-dihydrate. Three defined brain regions (Paxinos and Franklin, The mouse brain in Stereotaxic Coordinates. California: Elsevier Academic Press; 2004): the (1) prefrontal cortex (PFC), (2) hippocampus (Hic) and (3) hypothalamus (HYP), were immediately dissected and submerged in RNALater® (Qiagen Inc., USA) within 10 min of decapitation. Samples were initially stored at 4° C. overnight after which samples were moved to −20° C. for later processing according to manufacturer's instructions. All samples were collected within a 3.5 h window each day, starting at 7 h 30 and ending at 11 h 00. This window was defined to control for circadian fluctuations in hypothalamic-pituitary-adrenal (HPA) axis (HPAA) activity and associated stress susceptibility, in addition, basal HPAA activity is at a minimum during this window (Dalm et al., Neuroendocrinology; 2005; 81: 372-380). The individual age of mice at sample collection was approximately 93 days.

### PBMC Isolation

Using Optiprep™ (Axis-shield, Norway), a density floatation technique was employed to separate PBMC populations from whole blood samples. After collection of trunk blood, 250 μl aliquots were added to 12 ml sterile test tubes (Bibby Sterilin Ltd., UK), followed by 5 ml of a prepared tricine-buffered-saline (TBS)-lodixanol mixture (TBS: 0.85% NaCl, 10 mM Tricine, pH 7.4; TBS-iodixanol: 5 ml TBS and 1.5 ml Optiprep™). After mixing, an additional 0.5 ml TBS was gently layered on top of the blood-TBS-Optiprep™ mixture. Samples were centrifuged at 1000 g for 30 min at room temperature. PBMCs were collected, from the meniscus downward, in 4 ml of medium and added to a clean 12 ml tube. This suspension was diluted with two volumes of TBS. Cells were pelleted at 400 g for 10 min. The supernatant was carefully decanted, cells snap frozen in liquid N₂, and stored at −80° C. until further processing.

### Microarray Processing

### Experimental Design

Fifty-five samples, 15×PFC (8×MS and 7×SH), 10×Hic and 10×Hyp (5×MS and 5×SH, each and 20×PBMC (10×MS and 10×SH) were used for microarray processing, with a two-colour common reference design. Samples were matched, so that 10 individuals (5×MS and 5×SH) were completely represented in all tissues. A common reference pool was constructed by combining equal amounts (0.75 μg) of PFC and Hic RNA from both groups, which was stored as single aliquots of equal concentrations.

Commercial pre-spotted, full mouse genome, microarray slides (OpArray™) were sourced from Operon (Operon Biotechnologies, Germany), which were printed with version 4.0 of the Mouse Genome Oligo Set. This set contained 35,852 longmer probes, representing ±25,000 mouse genes and approximately 38,000 gene transcripts.

### RNA Purification, Quantification and Quality Assessments

All RNA purifications were performed using Qiagen RNeasy® kits (Qiagen Inc., USA). Neural tissues were processed using RNeasy® Lipid Tissue Mini solution and PBMC samples using the RNeasy® Mini solution. Samples were submerged in lysis buffers and frozen for 10 min prior to homogenisation. RNA extracts were quantitated and the purity (A₂₆₀/A₂₈₀ nm) 260-280 assessed using the Nanodrop ND-1000 spectrophotometer system (Nanodrop Technologies, USA). RNA integrity was determined using the Agilent BioAnalyzer 2100 System (Agilent, USA).

RNA amplification, labelling, hybridisation and image acquisition Due to limited amounts of starting material the Amino Allyl MessageAmp™ II aRNA amplification solution (Ambion Inc., USA) was employed to generate sufficient RNA quantities for microarray procedures. IVT incubation duration was 16 h at 37° C. for all samples (maximum recommended time was 14 h). All neural tissues were amplified from 0.5 μg total RNA, whereas all PBMC samples were amplified from 0.18 μg. Reference pool samples were amplified from 0.55 μg of total RNA, generating enough antisense RNA (aRNA) for ten hybridisations. All labelling reactions were done using 6.5 μg of aRNA. Reference aRNA was labelled with Cy5 and sample aRNA with Cy3™.

OpArray™ slides were prepared and processed according to the manufacturer's instructions. Hybridisations were performed using 170 ng of Cy3™ labelled sample and Cy5 labelled reference, at 42° C. for 16 h in humidified ArrayIt® hybridisation chambers (Telechem, USA). After washing, slides were dried by centrifugation at 200 g for 5 min. Slides were kept in a light protected air tight environment and scanned on the same day.

Images were acquired using an Axon 4000A dual-colour confocal laser scanner coupled to Genepix 6.0.27 Pro Software (Axon Instruments/Molecular Devices Corporation, CA, USA). Fluorescent signals were collected in Cy3 and Cy5 channels and quantified automatic morphological feature alignment and background estimation with manual adjustments where necessary. A predefined filter was used to flag features that failed to meet a set of minimum quality criteria.

Full details of RNA labelling, microarray hybridization, image capture and microarray data processing are given in Additional file 1: Supplementary Methods of Van Heerden et al. (2009), which is expressly incorporated herein (BMC Research Notes 2009, 2:195 doi:10.1186/1756-0500-2-195;

http://www.biomedcentral.com/content/supplementary/17560500-2-195-S1.DOC).
Microarray data are available in the ArrayExpress database (www.ebi.ac.uk/arrayexpress) under accession number E-MEXP-2101.

### Data Normalization

Data normalization was done in R, using the Limma package (Smyth, Smyth GK: Limma: linear models for microarray data. In Bioinformatics and Computational Biology Solutions using R and Bioconductor. Edited by Gentleman R et al.; 2005:397-420). All flagged features were down-weighted (to 0.001) during normalization, contributing minimally to correction factor estimations. Neural tissue hybridisations were normalized using Global Loess adjustments combined with between array normalization, using the median absolute deviation scaling method. PBMC samples were normalized using Print-tip Loess adjustments only, with default settings. Background adjustments were not performed on any of the hybridisations, as it was not found to improve the data. Normalization yielded log₂-transformed expression ratios, which were used for all subsequent procedures.

### Duplicate Merging, Missing Value Imputation and Removal of Batch Effects

Duplicate feature values were merged and missing values imputed using the Pre-processing module of GEPAS (Montaner et al.; Nucleic Acids Research 34 (Web Server issue), 2006, http://www.gepas.org). A first round of duplicate merging was done based on unique oligo identifiers (i.e. all features with the same oligo sequence). Following missing value imputation, a second round of duplicate merging was performed using primary gene identifiers (ENSEMBL Mouse release 43.36d, http://www.ensembl.orq; Refseq release 22, http://www.ncbi.nlm.nih.gov; or Riken release 3.0, http://fantom.gsc.riken.jp; where available and in order of preference), thus reducing the number of duplicate gene measurements. KNN imputation (with the default of 15 nearest neighbours) was used to estimate missing values for (1) PFC patterns with a minimum of 66% unflagged features (i.e. at least 10 out of 15 slides), (2) Hic and Hyp patterns with a minimum of 70% unflagged features (i.e. at least 7 out of 10 slides) and (3) PBMC patterns with a minimum of 55% unflagged features (i.e. 11 out of 20 slides).

Batch effects and other forms of structured noise were removed from data using ASCA-genes (Nueda et al., Bioinformatics 2007, 23(14):8). In the current study, a batch was defined as a single amplification, labelling, hybridisation and scanning run, which included five slides. Other sources of structured noise were identified as residual error and removed if any one factor accounted for more than four times the error that would be expected by chance.

### Differential Expression and Clustering

Differentially expressed genes were identified using a concordance strategy, based on overlap between three statistically divergent approaches. Genes that had a P-value <0.05, using the Info statistic (Kaminsky and Friedman, American Journal of Respiratory and Cell Molecular Biology, 2002, 27: 125-132), from the ScoreGenes software package (http://www.cs.huji.ac.il/labs/compbio/scoregenes/), and a P-value <0.05 using the Tusher at al. (Proc Natl Acad Sci USA 2001, 98(9):5116-5121) Significance Analysis of Microarrays (SAM) implementation in the T-Rex module of GEPAS (http://www.gepas.org), in addition to an absolute fold-change >1.2 (where fold change is defined as the fold difference between MS and SH) were considered to be differentially expressed (DE).

All data clustering was done in the Tigr MultiExperiment Viewer V4.1 (TMEV, http://www.tm4.org) from the TM4 suite of microarray analysis tools (Saeed et al., Biotechniques, 2003, 34: 374-378), using a Pearson correlation metric with average linkage.

### Sample Classification and Prediction

The efficiency of PBMC gene expression profiles at predicting the treatment class of samples (i.e. MS or SH) was evaluated with the Prophet module in GEPAS (Medina et al., 2007; http://www.gepas.org) using both the K-nearest neighbour (KNN) and Support Vector machine (SVM) algorithm options. Leave-one-out cross validation was used to counter selection bias whilst simultaneously assessing prediction efficacy.

### Results and Discussion

Microarray data comparing the response of control and MS adult mice to stress was used to investigate the presence of a functional link between gene expression changes in the brain and PBMCs. In the first instance data was analysed to characterise the transcriptional response of three brain regions, the prefrontal cortex, the hippocampus and hypothalamus to stress, and to investigate whether a co-ordinated change in glutamatergic and GABAergic systems occurred in MS mice. Corresponding differences in gene expression in PBMCs of MS mice compared to control mice were also identified. Importantly, these differences could be used to predict the treatment status of mice.

### Microarray Analysis

After normalization, replicate merging, removal of flagged features and imputation, the number of genes expressed in each tissue was: (1) PFC, 15 760; (2) Hic, 17 344; (3) Hyp, 15 794 and (4) PBMC, 13 306.

### MS Produced Gene Expression Differences in all Tissues

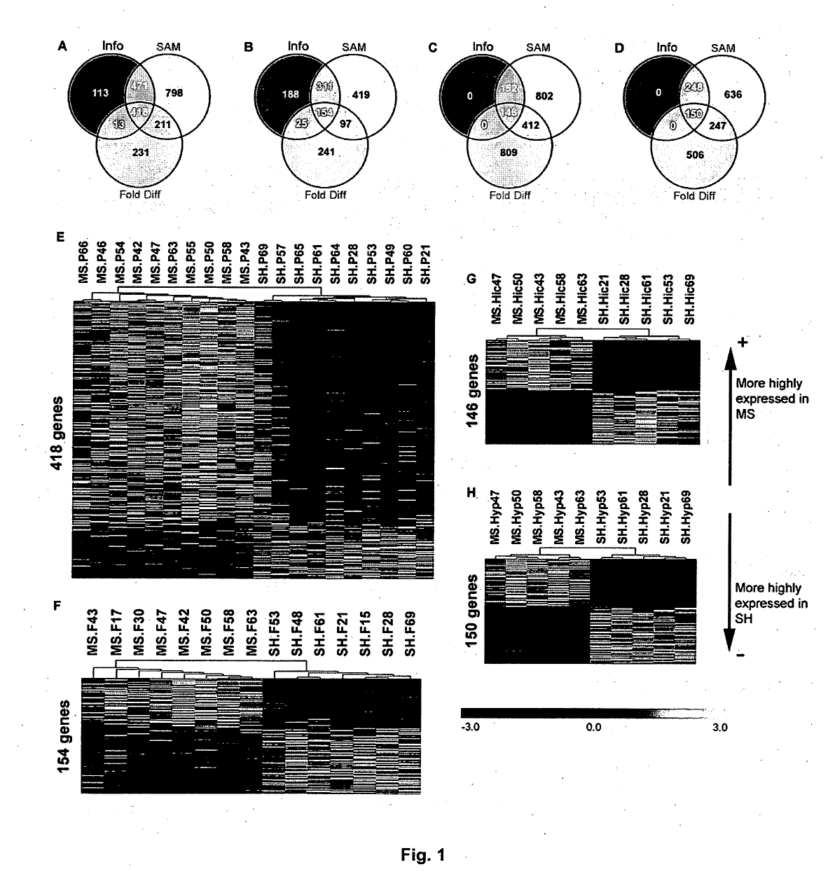

Differentially expressed (DE) genes were identified in all tissues (FIG. 1A-D). A summary of all DE genes is provided in a publication of this research by the inventors (BMC Research Notes 2009, 2:195 doi:10.1186/1756-0500-2-195, Table S2 [see Additional file 2; http://www.biomedcentral.com/content/supplementary/1756-0500-2-195-S2.XLS], Table S3 [see Additional file 3; http://www.biomedcentral.com/content/supplementary/1756-0500-2-195-S3.XLS], Table S4 [see Additional file 4; http://www.biomedcentral.com/content/supplementary/1756-0500-2-195-S4.XLS], and Table S5 [see Additional file 5; http://www.biomedcentral.com/content/supplementary/17560500-2-195-S5.XLS]). The unsupervised hierarchical sample clustering of differentially expressed genes, produced clear group (MS or SH) separations within all tissues (FIG. 1E-H). No single gene was differentially expressed across all tissues.

### Gene Set Enrichment Analysis Revealed Significant Functional Themes

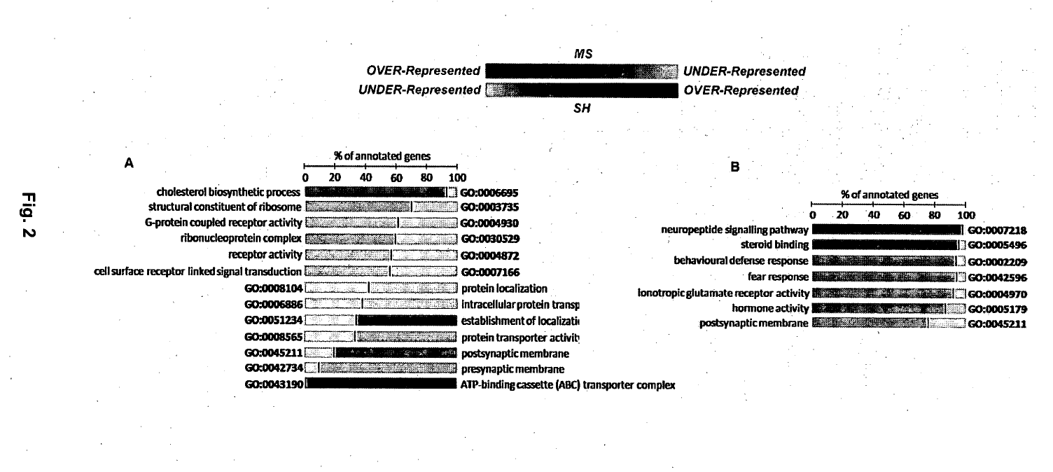

The FatiScan analysis revealed the significant enrichment of functional terms, in all tissues (FIG. 2). Interestingly, in PBMC samples (FIG. 2D), over-expressed terms could be grouped, generally, into signalling- (GO:0004872, GO:0051606, GO:0005887, GO:0007165, GO:0007154), immune- (GO:0006955, GO:0006952, GO:0005856, GO:0007275) and, interestingly, neurologically-related (GO:0008188, GO:0050877) classes. On the other hand, under-expressed terms all displayed a metabolic theme, with terms related to RNA and protein processing (GO:0003735, GO:0016070, GO:0044267, GO:0009058, GO:0009059, GO:0015031, GO:0006412, GO:0005840, GO:0003676 and GO:0043021) and energy metabolism (GO:0005739, GO:0051187 and GO:0006099). These results suggest a functional shift in the immune system in PBMCs in MS mice, characterised by the coordinated down-regulation of energy requiring processes, such as protein synthesis and transport. This functional shift might reflect the well characterised mobilisation of energy and inhibition of further storage in response to stress.

Response of the Glutamergic and GABergic Systems in Neural Tissues after Stress

DE genes and enriched functional terms from the PFC datasets highlighted the importance of the glutamatergic and GABAergic systems in the stress-related response of the MS mice. These two neurotransmitter systems constitute the major stimulatory (glutamate) and inhibitory (GABA) mechanisms of neurotransmission, and work counteractively to ensure optimal neuronal activity after stress. Glutamatergic signalling was enhanced in MS mice possibly as a consequence of deficiencies in GABAergic mediated inhibitory mechanisms.

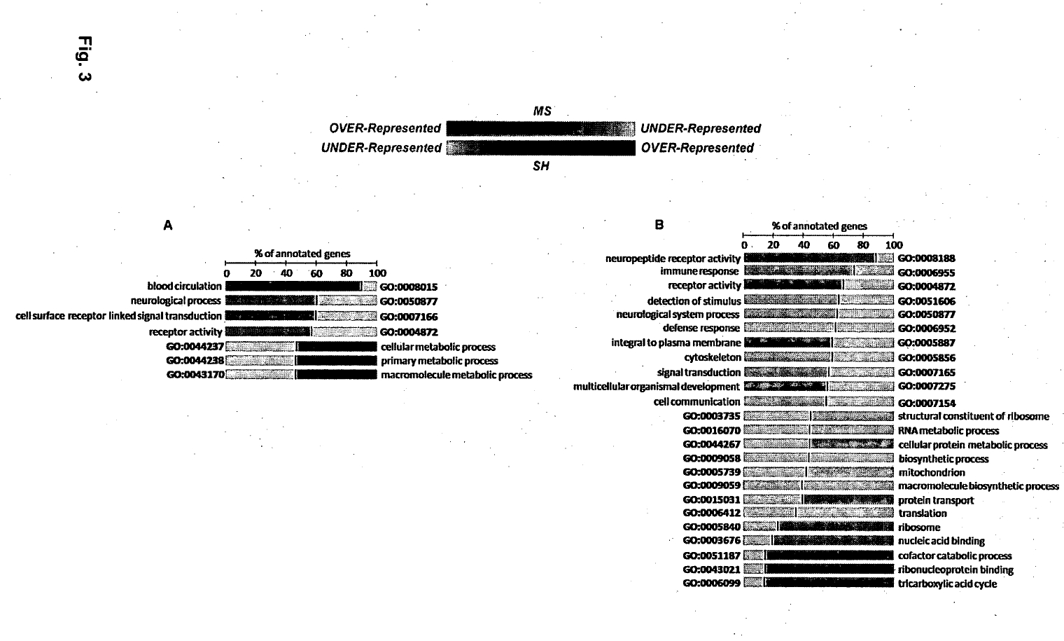

DE genes whose products are involved in the modulation of glutamatergic and GABAergic signalling included P2yr4 and Npvf (FIG. 3). The activation of P2yr4 positively regulates glutamate release, whereas Npvf is an important inhibitor of GABAergic neurotransmission. The over-expression of both these genes in the MS PFC tissue, points to a hyperactive glutamatergic system. Supporting this observation is the under-expression of Myo6 in the MS samples. Myo6 is crucial for the efficient endocytosis of postsynaptic glutamate receptors, with deficiencies resulting in increased excitatory neurotransmission. Htr3a was also under-expressed in MS samples. This receptor is strongly associated with GABAergic neurons and interneurons which activate the GABA mediated inhibitory neurotransmission in the prefrontal cortex. The co-ordinated under-expression of both pre- and post-synaptic component GO terms further supports the hypothesis of a hyperglutamatergic state in the PFC of MS mice (FIG. 3). Specifically, genes supporting depletion of postsynaptic components in MS mice included three GABA_{A} receptors (GABA_{A} alpha-1 and -3, and GABA_{A} gamma-3) (FIG. 3); such receptors mediate inhibition of neurotransmission with disruptions resulting in enhanced anxiety. Genes supporting functional depletion of presynaptic components included two metabotropic glutamate receptors, mGluR3 and mGluR7 (FIG. 3). These receptors participate in negative feedback mechanisms that inhibit presynaptic glutamate release. Results from the hippocampal gene expression dataset extend these observations, with the over-representation, in MS samples, of genes involved in ionotropic glutamate signalling (FIG. 3). Although this hyperglutamatergic theme was not readily apparent in either the DE genes or functionally enriched terms of the hypothalamus dataset, under-expression of cortistatin may be relevant insofar as cortistatin signalling inhibits glutamate induced responses in hypothalamus (FIG. 3).

### Functional Significance of Gene Expression Changes in PBMC Tissues

A large number of genes (418) were found to be differentially expressed between MS and SH individuals and included several genes whose products are important modulators of immune system function. Examples include Foxp3, an essential modulator of T cell function [23]; IL-17ra, the receptor target for the IL-17 mediated inflammatory pathway [24]; and Ccl5 (also known as Rantes), which regulates the activity of several cellular populations within the immune system.

The evidence obtained from the neural transcriptomes (combined with corticosterone and behavioural profiles; van Heerden et al Submitted Manuscript) indicates that pre-weaning treatment (MS or SH) result in differential stress-related profiles. Given this context, the gene expression information derived from the PBMC samples was evaluated in terms of its ability to derive accurate predictions of pre-weaning status of individuals.

### PBMC Gene Expression Profiles Accurately Predict Sample Classes

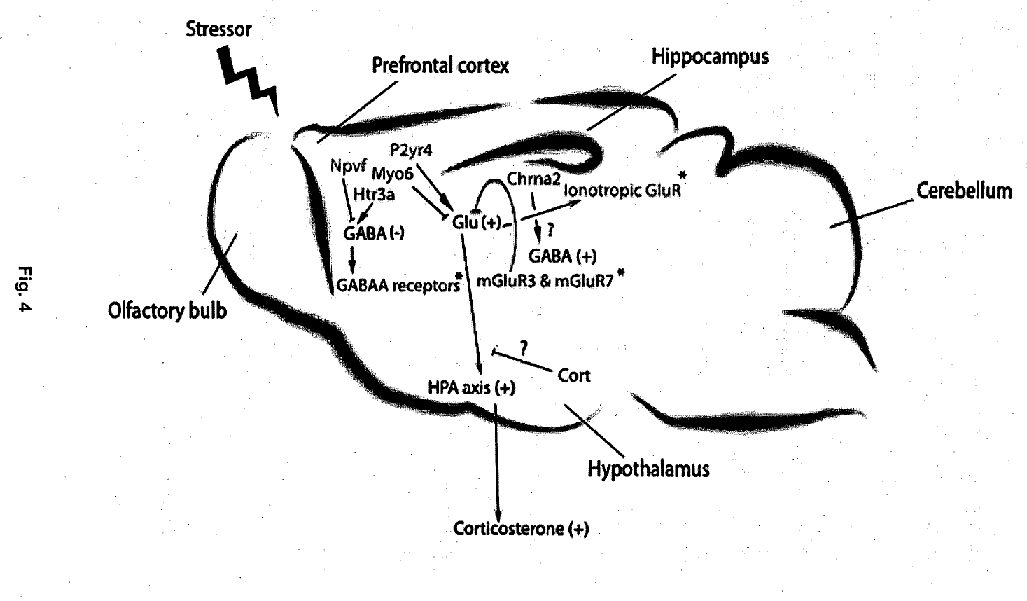

The classification and prediction of sample classes (MS or SH) using PBMC gene expression values, were found to be highly efficient. Using KNN (with 4 neighbours), 50 genes (FIG. 4; Table 1) were sufficient to accurately identify sample classes 19 out of 20 times. Most of the genes included in the predictor were over-expressed (FIG. 4B). SVM, however, only achieved this success rate using a minimum of 125 genes (with linear and radial kernels). Importantly, this 125 gene set (Table 2) consisted of the 50 genes included in Table 1, in addition to 75 other genes, which were the same for both algorithms (data not shown).

Of the 50 genes included in the predictor, 46 were functionally annotated. Of particular interest was the identification of 3 genes, Oxt, Cck and Adcy8 (all over-expressed), whose products are known to be important mediators of stress- and anxiety-associated behaviours (Table 1) [26] [27] [28]. Both Oxt and Cck are neuroactive hormones with previously described endogenous immunomodulatory properties. These results confirm that the transcriptional profiles of peripheral immune tissues do indeed contain sufficient information for the efficient diagnostic prediction of stress-related neural states in mice. Products of these genes may participate in pathways that are particularly sensitive to stress-induced regulation of the immune system.

1. A method of assessing a psychiatric disorder, a behavioral problem or a mental state in a subject following exposure to stress, the method comprising:
(a) determining the expression levels of at least 40 of the genes listed in Table 1 in a sample from the subject after said exposure;
(b) comparing the expression levels of said at least 40 genes with a reference set of expression levels for the same genes, thereby obtaining a set of comparison values, wherein a difference in the expression levels between that in (a) and the reference set is indicative of existence of, or susceptibility to the psychiatric disorder, behavioral problem or mental state in the subject.

2. The method according to claim 1, wherein the reference set comprises expression levels of the same genes in a healthy subject.

3. The method according to claim 1, wherein an algorithm is used to compare the expression levels of (a) with the reference set of (b), in generating the comparison values, and based on the comparison values, determining or predicting whether the subject has, or is susceptible to developing, the psychiatric disorder, behavioral problem or mental state.

4.-6. (canceled)

7. The method according to claim 1, further comprising, in step (a), determining in the subject expression levels of genes other than those in Table 1.

8. The method according to claim 7, wherein the other genes are selected from those listed in Table 2.

9. The method according to claim 8, wherein expression levels of at least 100 of the genes listed in Table 2 are determined.

10.-12. (canceled)

13. The method according to claim 1, wherein mRNA transcripts are used to determine the expression levels of the genes.

14. The method according to claim 1, wherein the sample is blood or a buccal smear or another bodily sample containing white blood cells.

15. (canceled)

16. The method according to claim 1, wherein peripheral blood mononuclear cells (PBMCs) are isolated from the subject's sample.

17. The method according to claim 1, wherein step (a) is performed on RNA or mRNA transcripts isolated from the subject's sample.

18. The method according to claim 1 wherein step (a) is performed using quantitative reverse-transcriptase polymerase chain reaction (RT-qPCR) by Real-Time PCR.

19. (canceled)

20. The method according to claim 1, wherein step (a) is performed using a dot blot procedure.

21. The method according to claim 1 wherein step (a) is performed using a microarray printed with oligonucleotides corresponding to at least 40 of the genes listed in Table 1, 100 of the genes listed in Table 2, or both.

22. The method according to claim 1, which includes the steps of:
(a) isolating PBMCs from whole blood of the subject;
(b) purifying total RNA from the PBMCs;
(c) amplifying, by RT-qPCR of said total RNA, mRNA transcripts of at least 40 of the genes listed in Table 1, at least 100 of the genes listed in Table 2, or both;
(d) quantifying the amplified mRNA transcripts;
(e) assessing relative abundance of the amplified mRNA transcripts of each of the genes; and
(f) using the mRNA transcript abundances assessed in step (e) with a classification and prediction algorithm to classify the psychiatric disorder, behavioral problem, or mental state of the subject as normal or pathological.

23. The method according to claim 1, which includes the steps of:
(a) isolating PBMCs from whole blood of the subject;
(b) purifying the total RNA from the PBMCs;
(c) converting the RNA into cDNA by reverse transcription and labelling the cDNA with a fluorescent dye or a chromogenic substrate;
(d) hybridizing the labelled cDNA to a dot blot printed with probes corresponding to at least 40 of the genes listed in Table 1, at least 100 of the genes listed in Table 2, or both;
(e) quantifying the intensity of the hybridized labelled cDNA, the intensity of the label corresponding to the relative abundance of mRNA transcripts; and
(f) using the mRNA transcript abundances obtained in step (e) with a classification and prediction algorithm to classify the psychiatric disorder or mental state of the subject as normal or pathological.

24. The method according to claim 1, which includes the steps of:
(a) isolating PBMCs from whole blood of the subject;
(b) purifying total RNA from the PBMCs;
(c) converting RNA into cDNA by reverse transcription, and labelling the cDNA with a fluorescent dye to generate fluorescent cDNA;
(d) contacting a microarray slide printed with oligonucleotide probes representing at least 40 genes listed in Table 1, at least 100 genes listed in Table 2, or both, with the fluorescent cDNA;
(e) hybridizing the fluorescent cDNA to the oligonucleotide probes representing said genes printed on the microarray slide and quantifying the fluorescence intensity of the fluorescent cDNA bound to target oligonucleotides on the slide, the intensity of fluorescence at each target corresponding to the relative abundance of mRNA transcripts for each gene; and
(f) using the mRNA transcript abundances obtained in step (e) with a classification and prediction algorithm to classify the psychiatric disorder or mental state of the subject as normal or pathological.

25. A kit for use in the method of claim 1, comprising:
(a) primers for amplification of mRNA transcripts by RT-qPCR of at least 40 of the genes listed in Table 1, at least 100 of the genes listed in Table 2, or both; or
(b) probes printed on a membrane for hybridizing mRNA transcripts of at least 40 of the genes listed in Table 1, at least 100 of the genes listed in Table 2, or both, for use in an RNA dot blot procedure; or
(c) oligonucleotides corresponding to at least 40 of the genes listed in Table 1, at least 100 of the genes listed in Table 2, or both, printed on a slide, to which fluorescent cDNA can be hybridized when a microarray is used in the method; and
(d) an indicator means, and
(e) optionally, instructions for performing the method.

26. (canceled)

27. The kit according to claim 25, which is for use in an RNA dot blot process, a miniaturized dot blot process or for a microarray platform.

28.-29. (canceled)

30. The kit according to claim 25, which further includes a reference set of expression levels for at least 40 of the genes listed in Table 1, at least 100 of the genes listed in Table 2, or both, which reference set comprises expression levels of the genes from a healthy or normal subject.

31. The kit according to claim 25, which includes computer readable instructions for:
(a) comparing expression levels of the genes from a subject with a reference set of expression levels for the same genes, which results in a set of comparison values; and
(b) predicting whether the subject has, or is susceptible to developing, a psychiatric disorder, a behavioral problem or a mental state; and

32. The kit according to claim 31 which further includes computer readable instructions for:
(c) generating a data set from the comparison values of (a),which data set indicates whether or not the subject has, or is susceptible to developing, said psychiatric disorder, behavioral problem or mental state.

33. The method according to claim 1, further comprising, after step (b), a step of:
(c) generating a data set from the expression levels of (a) and the comparison values of (b), which data set indicates whether or not the subject has, or is susceptible to developing, said psychiatric disorder, behavioral problem or mental state.

34. A method for deciding whether to treat a subject for the presence of, or susceptibility of developing, a psychiatric disorder, a behavioral problem or a mental state in response to stress exposure, comprising
(a) assessing the data set generated by the method of claim 1 for the subject, and
(b) determining whether the subject has or is susceptible to said disorder problem or state,
wherein, a decision to treat follows from determining said presence or susceptibility.

35. A method for deciding whether to treat a subject for the presence of, or susceptibility of developing, a psychiatric disorder, a behavioral problem or a mental state in response to stress exposure, comprising
(a) assessing the data set for a subject generated using the kit according to claim 31; and
(b) determining whether the subject has, or is susceptible to, said disorder problem or state,
wherein, a decision to treat follows from determining said presence or susceptibility.
